(19) United States
(12) Patent Application Publication
(10) Pub. No.: US 2026/0011446 A1
(43) Pub. Date: Jan. 8, 2026

(54) METHOD AND SYSTEM FOR DIGITAL ADMINISTRATION OF PTSD TREATMENT

(71) Applicant: SAVYN Tech Inc., Toronto (CA)

(72) Inventors: Sakeena Mihar, Toronto (CA); Vic Shao-Chih Chiang, Auckland (NZ)

(21) Appl. No.: 18/765,213

(22) Filed: Jul. 5, 2024

(51) Int. Cl. G16H 50/30; A61H 5/00; A61M 21/00

(52) U.S. Cl. CPC G16H 50/30; A61H 5/00; A61M 21/00; A61M 2021/005

(57) ABSTRACT

A digital treatment program for trauma and PTSD is disclosed along with the methods implemented by the program. This program, method and protocol include pre- and post-treatment assessments to measure progress and detect potential problems, and includes a number of eye movement and exposure therapy sessions. Throughout the sessions, the patient is enabled to access grounding tools, and then optionally return to the session, which reduces the difficulties faced by the patient while going through the protocol, promoting completion of the protocol. The assessments, responses and progress of the patient are recorded and stored in a database and can be accessed by a caregiver, who monitors the treatment and progress by the patient. Apparatus is also disclosed to implement the digital treatment program.

### FIELD OF THE INVENTION

The improvements generally relate to the field of supervision of therapy by a caregiver for patients with trauma and PTSD (post-traumatic stress disorder).

### BACKGROUND OF THE INVENTION

Traumatic events can cause post-traumatic stress disorder (PTSD). PTSD is a serious condition, and symptoms can include anxiety, flashbacks to the traumatic event(s), nightmares, and generally uncontrollable and negative thoughts about the traumatic event(s) and the person suffering from PTSD. One study (Steel et al., 2009) estimates that 70% of the global population has been exposed to trauma at least once in their life, and 13% of these individuals develop PTSD. There is a need for new approaches to the treatment of PTSD.

There are published approaches to remote therapy for PTSD. Examples include U.S. Pat. No. 11,830,628 (Thorn et al.), U.S. Pat. No. 11,865,268 (Gazit and Gazit), US2018/0177973 (Keene et al.) and US2022/0230766 (Lowery-Luyties et al.).

### INCORPORATION BY REFERENCE

Each cited patent, publication, and non-patent literature is hereby incorporated by reference in its entirety, as if each was incorporated by reference individually, and as if each is fully set forth herein. However, no such citation should be construed as an admission that a cited reference comes from an area that is analogous or directly applicable to the invention, nor should any citation be construed as an admission that a document or underlying information, in any jurisdiction, is prior art or forms part of the common general knowledge in the art.

### BRIEF SUMMARY OF THE INVENTION

The following presents a simplified summary of some embodiments of the invention in order to provide a basic understanding of the invention. This summary is not an extensive overview of the invention. It is not intended to identify key or critical elements of the invention or to delineate the scope of the invention. Its sole purpose is to present some embodiments of the invention in a simplified form as a prelude to the more detailed description that is presented later.

It is desirable, in order to allow wider and easier access to therapy to address trauma and PTSD, to have a digital system whereby a caregiver can supervise access to therapy by a patient experiencing trauma and PTSD. In some cases, it is desirable for the system to allow the patient to be remote from the caregiver.

The invention is a method and system to be used by a caregiver to administer treatments digitally and effectively for patients with trauma and PTSD. Apparatus to provide access to the digital treatment program are also described. The inventors have created a unique and inventive digital protocol (tested through studies) suited for the administration of eye movement and exposure therapy and implemented as a digital treatment program. These methods and access to the program can be implemented in off-line situations where the patient does not have access to the Internet (or sporadic or unreliable access) through the implementation of the methods in a digital treatment program on a local computer or laptop. In the case of a laptop, the methods can also be used in areas where power is sporadic or non-existent. In one preferred embodiment, the administration is remote in that the caregiver is remote from the patient. In a preferred embodiment, this can be done through a software as a service framework.

The disclosed methods, protocols and systems in some aspects provides improvements over previous administration methods and systems, and in some aspects can provide improvements over in-person administrations of treatments.

In accordance with the present invention, there is provided a method for the administration of therapy to a patient supervised by a caregiver, comprising: administering assessments to the patient to qualify them to undertake the treatment; administering assessments to the patient to assess their pre-treatment condition; taking a detailed history of traumatic events; having the patient set up a grounding tools; and for a specific traumatic event: administering a pre-treatment check of the patient's receptiveness to treatment, by administering a first assessment and a second assessment, and stopping treatment if a first predetermined threshold is not met on these assessments; administering an eye movement treatment comprising a cursor moving left to right and back across a computer screen; inquiring as to whether the patient has changed feelings from the treatment, and if the answer is yes administering a third assessment, and if the third assessment meets a second predetermined threshold, notifying the caregiver; where at all times after the step of setting up grounding tools, the patient has the ability to exit the treatment method and access grounding tools, and optionally can return to the treatment method after accessing the grounding tools.

In an aspect of the invention, the method further comprises: for the specific traumatic event: administering an exposure therapy pre-treatment check of the patient's receptiveness to treatment, by administering a fourth assessment and a fifth assessment, and stopping treatment if a third predetermined threshold is not met on these assessments; administering an exposure therapy treatment comprising the patient recording their thoughts regarding the traumatic event; inquiring as to whether the patient has changed feelings from the exposure therapy treatment, and if the answer is yes administering a sixth assessment, and if the sixth assessment meets a fourth predetermined threshold, notifying the caregiver; where during the steps of administering an exposure treatment, and inquiring as to whether the patient has changed feelings from the exposure therapy treatment, the patient has the ability to exit the treatment method and access grounding tools, and optionally can return to the treatment method after accessing the grounding tools.

In another aspect of the invention, the method further comprises repeating the steps of administering a pre-treatment check; administering an eye movement treatment, and inquiring as to whether the patient has changed feelings from the treatment, six to eight times.

In still another aspect of the invention, the method further comprises: repeating the steps of administering a pre-treatment check; administering an eye movement treatment, and inquiring as to whether the patient has changed feelings from the treatment, six to eight times; and repeating the steps of administering an exposure therapy pre-treatment check; administering an exposure treatment, and inquiring as to whether the patient has changed feelings from the exposure therapy treatment, three to five times.

In another aspect of the invention, the second, third, fifth and sixth assessments are SUDS assessments. In still another aspect of the invention, the first and fourth assessments are PCL assessments. In another aspect of the invention, the patient and caregiver are remote.

In accordance with the present invention, there is provided a system for the administration of therapy to a patient supervised by a caregiver, comprising: a first computing device, the first computing device being configured to: administer assessments to the patient to qualify them to undertake the treatment; administer assessments to the patient to assess their pre-treatment condition; take a detailed history of traumatic events; have the patient set up a grounding tools; and for a specific traumatic event: administer a pre-treatment check of the patient's receptiveness to treatment, by administering a first assessment and a second assessment, and stopping treatment if a first predetermined threshold is not met on these assessments; administer eye movement treatment comprising a cursor moving left to right and back across a computer screen; inquire as to whether the patient has changed feelings from the treatment, and if the answer is yes administering a third assessment, and if the third assessment meets a second predetermined threshold, notifying the caregiver; where at all times after the step of setting up grounding tools, the patient has the ability to exit the treatment method and access grounding tools, and optionally can return to the treatment method after accessing the grounding tools.

In an aspect of the invention, the system further comprises the first computing device is further configured to: for the specific traumatic event: administer an exposure therapy pre-treatment check of the patient's receptiveness to treatment, by administering a fourth assessment and a fifth assessment, and stopping treatment if a third predetermined threshold is not met on these assessments; administer an exposure therapy treatment comprising the patient recording their thoughts regarding the traumatic event; inquire as to whether the patient has changed feelings from the exposure therapy treatment, and if the answer is yes administering a sixth assessment, and if the sixth assessment meets a fourth predetermined threshold, notifying the caregiver; where during the steps of administering an exposure treatment, and inquiring as to whether the patient has changed feelings from the exposure therapy treatment, the patient has the ability to exit the treatment method and access grounding tools, and optionally can return to the treatment method after accessing the grounding tools.

In another aspect of the invention, the system, further comprises the first computing device being configured to: repeat the steps of administering a pre-treatment check; administer an eye movement treatment, and inquire as to whether the patient has changed feelings from the treatment, six to eight times.

In still another aspect of the invention, the system, further comprises the first computing device being configured to: repeat the steps of administering a pre-treatment check; administer an eye movement treatment, and inquire as to whether the patient has changed feelings from the treatment, six to eight times; and repeat the steps of administering an exposure therapy pre-treatment check; administering an exposure treatment, and inquiring as to whether the patient has changed feelings from the exposure therapy treatment, three to five times.

In another aspect of the invention, the second, third, fifth and sixth assessments are SUDS assessments. In still another aspect of the invention, the first and fourth assessments are PCL assessments.

In another aspect of the invention, the system further comprises the first computing device being configured by the first computing device executing a program, and further comprising a second computing device, and the patient accessing the program being executed on the first computing device by communication between the second computing device and the first computing device.

In still another aspect of the invention, the system further comprises the first computing device being configured by the first computing device executing a program, and further comprising a third computing device, and the caregiver accessing the program being executed on the first computing device by communication between the third computing device and the first computing device.

The foregoing has outlined broadly and in summary certain pertinent features of the disclosure so that the detailed description of the invention that follows may be better understood, and so that the present contribution to the art can be more fully appreciated. Hence, this summary is to be considered as a brief and general synopsis of only some of the objects and embodiments disclosed herein, is provided solely for the benefit and convenience of the reader, and is not intended to limit in any manner the scope, or range of equivalents, to which the claims are lawfully entitled. Additional features of the invention are described hereinafter. It should be appreciated by those in the art that all disclosed methods are only exemplary, and may be readily utilized as a basis for modifying or designing other methods for carrying out the same purposes. Such equivalent methods will be appreciated to be also within the scope and spirit of the invention as set forth in the claims.

The headings within this document are being utilized only to expedite its review by a reader. They should not be construed as limiting the invention in any manner.

### BRIEF DESCRIPTION OF THE DRAWINGS

Embodiments will now be described, by way of example only, with reference to the attached figures, wherein in the figures:

FIG. 1 depicts a flowchart of the overall method;

FIG. 2 depicts a flowchart of each eye movement session;

FIG. 3 depicts a flowchart of each exposure therapy session;

FIG. 4 depicts a first setup of a computing system to implement the digital treatment program and method.

FIG. 5 depicts a second setup of a computing system to implement the digital treatment program and method.

### DETAILED DESCRIPTION OF THE INVENTION

While various aspects and features of certain embodiments are summarized above, the following detailed description illustrates several exemplary embodiments in further detail to enable one of skill in the art to practice such embodiments, and to make and use the full scope of the invention claimed. The described examples are provided for illustrative purposes and are not intended to limit the scope of the invention or its applications. It will be understood that many modifications, substitutions, changes, and variations in the described examples, embodiments, applications, and details of the invention illustrated herein can be made by those skilled in the art without departing from the spirit of the invention, or the scope of the invention as described in the appended claims.

As used in this specification and the appended claims, the singular forms “a,” “an,” and “the” include plural referents unless the context clearly dictates otherwise. While the term “one or more” may be used, its absence (or its replacement by the singular) does not signify the singular only, but simply underscores the possibility of multiple agents or ingredients in particular embodiments.

The terms “comprising,” “including,” “such as,” and “having” are intended to be inclusive and not exclusive (i.e., there may be other elements in addition to the recited elements). Thus, the term “including” as used herein means, and is used interchangeably with, the phrase “including but not limited to.” The term “or” is used herein to mean, and is used interchangeably with, the term “and/or,” unless context clearly indicates otherwise.

Generally, the nomenclature used and procedures performed herein are those known in fields relating to one or more aspects of the invention, such as computer science, software engineering, psychiatry, psychology, and the like, and are those that will be well known and commonly employed in such fields. Standard techniques and procedures will be those generally performed according to conventional methods in the art.

Unless defined otherwise, all technical and scientific terms herein have the meaning as commonly understood by one having ordinary skill in the art to which this invention belongs, who as a shorthand may be referred to simply as “one of skill.”

Further definitions that may assist the reader in understanding the disclosed embodiments are as follows; however, it will be appreciated that such definitions are not intended to limit the scope of the invention, which shall be properly interpreted and understood by reference to the full specification (as well as any plain meaning known to one of skill in the relevant art) in view of the language used in the appended claims. The terminology used herein is for the purpose of describing particular embodiments only and is not intended to be limiting.

Still additional definitions and abbreviations are provided elsewhere herein.

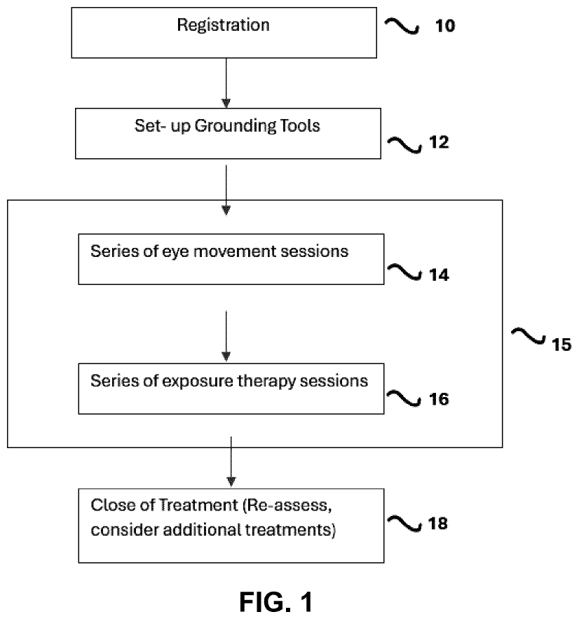

A flowchart of the treatment method is illustrated in FIG. 1.

In one preferred embodiment, each step of FIG. 1 is implemented on a SaaS basis, with the patient remotely logging in and accessing the treatment. In another embodiment, the patient accesses the treatment program through a computer with the steps in FIG. 1 implemented locally, and the caregiver is remote to the computer or server hosting the program. In another embodiment, the treatment and program are accessed by both the patient and caregiver locally.

Throughout this application, the term “computer” includes laptops, tablets, and other computing devices.

Turning to FIG. 1, there is first a registration step 10, where the patient creates an account for the software, and is associated with a caregiver.

The patient then completes an assessment and setup step 12.

During the first two steps 10 and 12, the patient: completes a number of assessments to qualify as an eligible candidate for treatment using this program. These assessments measure well-being, trauma and non-trauma related symptoms. Such assessments include the PCL (Post-traumatic Stress Disorder Checklist) and SUDS (Subjective Units of Distress) assessments, and can include the DES (Dissociative Experiences Scale), ACE (Adverse Childhood Experience), GAD (Generalized Anxiety Disorder), and PHQ (Patient Health Questionnaire) assessments, PTCI (Post-Traumatic Cognitions Inventory), GAF (Global Assessment of Functioning), MBI (Maslach Burnout Inventory), STSS (Secondary Traumatic Stress Scale), BRS (Brief Resilience Scale), or CFQ (Cognitive Failures Questionnaire). A person skilled in the art will know of other assessments that could be used. The patient also completes a detailed history which includes an identification of past traumatic events. In a preferred embodiment, these assessments and history are passed to the caregiver, who decides whether the patient should proceed with treatment. In another embodiment, the decision to proceed is automated, based on the assessments meeting a minimum score (which, in another embodiment, can be set by the caregiver, and in another embodiment, is set by the person who sets up the treatment software).

Also as part of steps 10 and 12, the patient sets up grounding tools that are tools a patient can use to calm and ground themselves if they become upset or triggered. (Triggered generally means the patient experiencing negative out-of-control thoughts about trauma or PTSD that are out of control in that the patient cannot stop thinking about them or return to a calm state. A person skilled in the art will be familiar with this term.) A person skilled in the art will be familiar with several grounding tools. Among these tools are breathing exercises, a calm place and a container. A calm place is a detailed place (for example, a location that the patient knows, with details of its appearance and features, where the patient feels relatively safe and calm) that the patient can visualize to calm themselves.

A container is a safe mental space for the patient to place their thoughts and emotions in order to put those thoughts and emotions temporarily out of their mind.

Usually the above steps are completed in the first two sessions where the second session is roughly a week after the initial session. In a preferred embodiment, step 12 is implemented during the second session. In a less preferred embodiment, steps 10 and 12 are done during one session.

To proceed past step 12 to step 15, a caregiver has to consent to acting as a caregiver for each patient, and that caregiver is associated with this specific patient. The caregiver, among other roles, schedules the remaining sessions for the treatment program, with a session occurring every 6 to 8 days. Throughout the method, the caregiver has access to the information that is inputted into the system by the patient (whether written or verbal recordings), including assessments and results of assessments, and records created by the patient.

In step 15, the patient goes through sessions illustrated as steps 14 and 16.

For steps 14 and 16, the patient always has the option of leaving the treatment protocol and moving to grounding tools.

In step 14, the patient undergoes a series of eye movement sessions. In step 16, the patient undergoes a series of exposure therapy sessions. These steps 14 and 16 address a specific trauma (generally identified in the patient's detailed history).

In one embodiment, the patient is administered only the eye movement therapy in step 14, and step 16 is omitted. In another embodiment, the patient is administered only the exposure therapy in step 16, and step 14 is omitted. However, the preferred embodiment is to administer both step 14 and step 16.

In another embodiment, the sessions in step 15 (which includes steps 14 and 16) can be a mixture of the eye movement sessions and the exposure therapy sessions. For example, a patient could go through two eye movement sessions, then do an exposure therapy session, and then proceed with another eye movement session.

In a preferred embodiment, the patient undergoes a series of 6 to 8 eye movement sessions. In a preferred embodiment, the patient undergoes a series of 3 to 5 exposure therapy sessions.

In step 18, the patient follows the exit protocol. The patient repeats assessments to check progress. In a preferred embodiment, the assessment is a PCL assessment. The caregiver has the option of scheduling a repeat of step 15 (including steps 14 and 16), and in a preferred embodiment step 15 is applied to a different trauma of the patient.

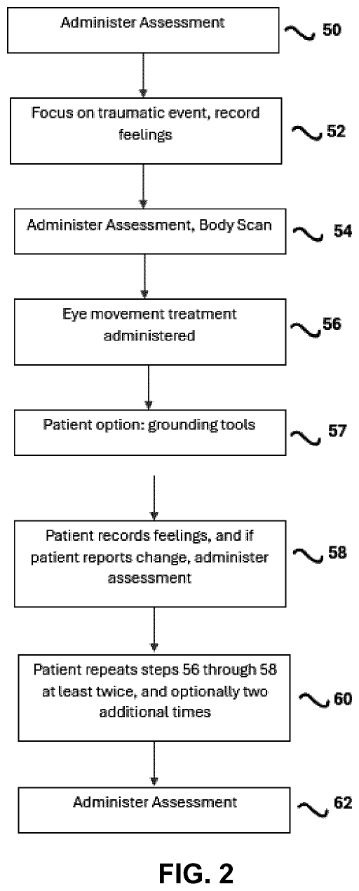

FIG. 2 details step 14: a series of eye movement sessions. Turning to FIG. 2, in step 50 an assessment is administered, and the patient must pass a set threshold to proceed with the session. In a preferred embodiment, the threshold is set by the caregiver. In another embodiment, the threshold is set by the administrator of the program. If the patient is not allowed to proceed with the session, the patient will return the following week to attempt the session again. In a preferred embodiment, the assessment is a PCL test.

In step 52, the patient focuses on a traumatic event (in a preferred embodiment, the patient focuses for at least around 15 seconds (where around means plus or minus 2 seconds)), and records their feelings about the traumatic event. (This traumatic event should be the same throughout step 15 (steps 14 and 16) from FIG. 1). In step 54, the patient performs a second assessment, and a body scan. In a preferred embodiment, the second assessment is a SUDS assessment. A body scan encourages the patient to focus on physical sensations associated with their present feelings and record their impressions. In step 56, the eye movement treatment is administered: while holding their head still, the patient follows a cursor or image left and right across the computer screen while thinking about the traumatic event. In a preferred embodiment, the eye treatment has a duration of around 23-24 seconds (where around means plus or minus 2 seconds). In step 57, the patient may optionally move to their grounding tools. In step 58, the patient records how they are feeling now (post-treatment, which may cause the patient to be triggered), and if they report changes in their feelings a third assessment is administered. In a preferred embodiment, this third test is a SUDS test. In another preferred embodiment, the second and third assessments are the same assessment type. In step 60, the patient is re-directed back to steps 56 through 58 twice more for a total of three administrations of eye movement (in step 56), and thereafter the patient can optionally choose to repeat steps 56 through 58 two more times. After the last iteration of steps 56 and 58, in step 62 an assessment is administered to the patient to check progress. In a preferred embodiment, this assessment is a PCL assessment.

After the very last eye movement treatment session, assessment(s) are administered to the patient as an additional measure of overall progress. In a preferred embodiment, one assessment is the GAD. In another preferred embodiment, one assessment is the PHQ.

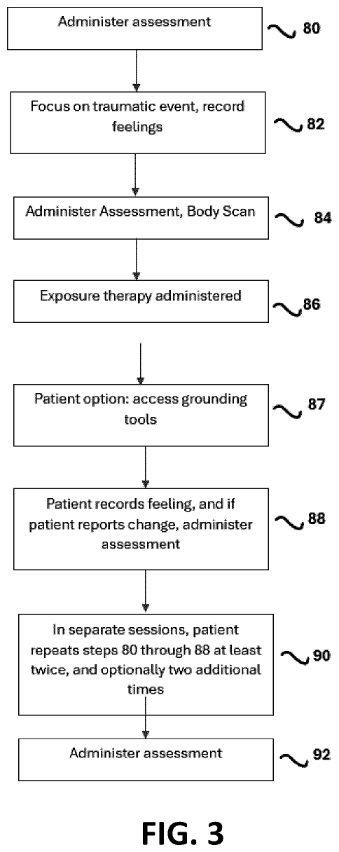

FIG. 3 details step 16: a series of exposure therapy sessions. Turning to FIG. 3, in step 80 an assessment is administered, and the patient must pass a set threshold to proceed with the session. In a preferred embodiment, the assessment is a PCL. In a preferred embodiment, the threshold is set by the caregiver. In another embodiment, the threshold is set by the administrator of the program. If the patient is not allowed to proceed with the session, the patient will return next week to attempt the session again.

In step 82, the patient focuses on a traumatic event for at least around 15 seconds, and records their feelings about the event (where around means plus or minus 2 seconds). (This event should be the same throughout steps 14 and 16 from FIG. 1). In step 54, the patient performs a second assessment, and a body scan. In a preferred embodiment, the second assessment is a SUDS assessment.

In step 86, the patient writes about the traumatic event. In step 87, the patient may optionally access their grounding tools. Once the patient is finished, in step 88 a third assessment is administered to measure progress. In a preferred embodiment, the third assessment is a SUDS assessment. In a preferred embodiment, the second assessment and the third assessment are the same assessment type.

Throughout steps 80-88, the patient optionally can engage their grounding tools.

In step 90, the exposure therapy discussed in steps 80 through 88 are repeated in separate sessions 3 to 5 times, as chosen by the caregiver (often the caregiver will consult with the patient for this choice). In step 92, once the exposure therapy sessions are completed, an assessment is administered to check progress. In a preferred embodiment, the assessment is a PCL assessment.

The invention also requires a separate module(s) to handle registration and control of the system by a caregiver. The caregiver schedules appointments, and in doing so decides what next steps are for the patient (for example, if based on the assessments the patient is not ready for eye movement treatment, then the caregiver will not schedule any sessions). The caregiver also has access to all assessments, and records made by the patient, and records of the patient's progress through the sessions, and the caregiver can intervene if they believe intervention to be desirable.

The system automatically alerts the caregiver if the assessment administered either at the beginning or the end of each treatment session (i.e. each of the eye movement treatment sessions in step 14 or each of the exposure therapy sessions in step 16) falls below a certain threshold. In a preferred embodiment, the threshold is set by the caregiver. In another embodiment, the threshold is set by the administrator of the program.

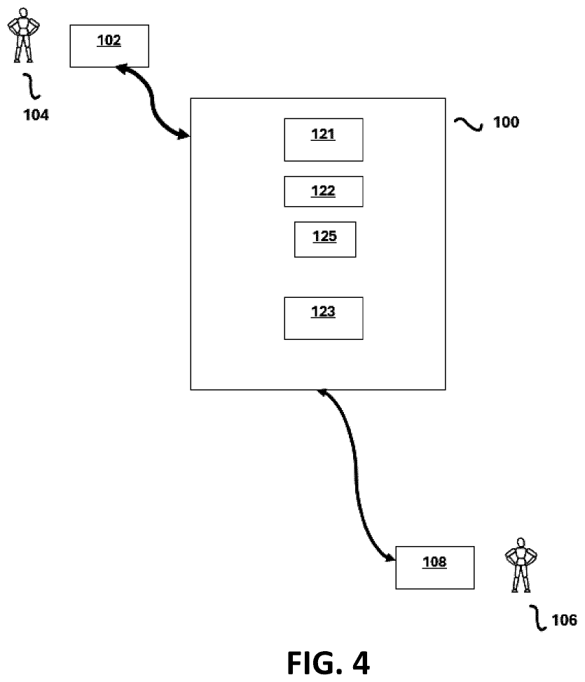

FIG. 4 illustrates the parts of a system to implement the methods of FIGS. 1-3. Turning to FIG. 4, there is a server 100. In a preferred embodiment, computer/server 100 is in communication with remote computer 102 and remote computer 108. The digital treatment program disclosed herein resides on computer/server 100 and is accessed by a patient 104 via remote computer 102. In operation, patient 104 logs in to the program residing on computer/server 100, and computer/server 100 sends and receives information from remote computer 102. There is also caregiver 106 who logs in to the digital treatment program residing on computer/server 100 via remote computer 108.

Computer/server 100 includes a processor 121, non-transitory computer-readable storage medium or memory 122 (which can include a database), and interface unit 123 (that interfaces between the computers 102 and 108 and computer/server 100), and the processor 121 and memory 122. Server 100 executes computer instructions 125 constituting a digital treatment program. In operation, information from the patient (including assessments, recordings by the patient whether in text or verbal, and other information input by the patient) is stored by server 100, and in a preferred embodiment in a database. In one embodiment, the caregiver 106 can download some or all of this information to computer 108 for the caregiver's use. In another embodiment, the patient 104 can download some of all of this information to computer 102 for the patient's use.

In another embodiment, the caregiver 106 accesses the digital treatment program directly through computer/server 100, and so remote computer 108 is not needed. In another embodiment, the patient 104 accesses the digital treatment program directly through computer/server 100, and so remote computer 102 is not needed.

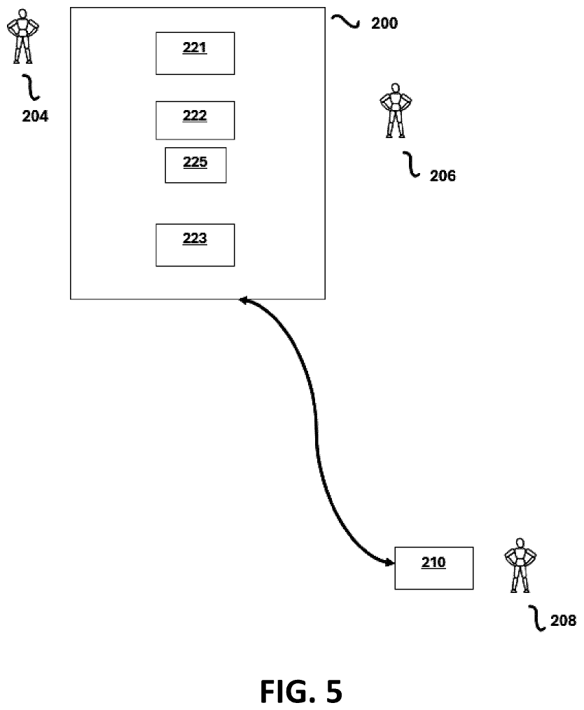

In another embodiment, computer 102 and patient 104 is in a location where access to the Internet or other communication methods is sporadic or non-existent, Computer 102 and patient 104 can also be in a location where there is no power, or sporadic power. In such cases, as shown in FIG. 5, the patient 204 accesses the digital treatment program through computer 200, which can be a laptop for portability or for use in cases where a lack of power is a concern. Turning to FIG. 5, patient 204 accesses a digital treatment program 225 on computer 200. Computer 200 includes a processor 221, non-transitory computer-readable storage medium or memory 222 (which can include a database), and interface unit 223, and in the case of a computer 210 interfaces between computer 200 and computer 210. Computer 200 includes executable computer instructions 225 constituting a digital treatment program. In operation, information from the patient (including assessments, recordings by the patient whether in writing or verbal, and other information input by the patient) is stored by digital treatment program 225, and in a preferred embodiment in a database.

The caregiver 206 can also access the digital treatment program 225 through computer 200, since the patient's responses, assessments and other records are stored in computer 200.

In another embodiment also illustrated in FIG. 5, there is a caregiver 208 who is remote from patient 204 and computer 200. Caregiver 208 can access digital treatment program 225 through computer 210, as long as there is at least intermittent communication between computer 210 and computer 200. In operation, information from the patient (including assessments, recordings by the patient whether in writing or verbal, and other information input by the patient) is stored by digital treatment program 225 in computer 200. In one embodiment, the caregiver 208 can download some or all of this information to computer 210 for the caregiver's use.

In all the embodiments discussed above in relation to FIGS. 4 and 5, the computers can be computers, laptops, tablets, or other computing devices. Input and output components, such as keyboards, cameras, microphones, speakers and screens are not illustrated but will be known to a person skilled in the art.

A person skilled in the art will know that the references to servers can include cloud servers as well as physical servers. In one embodiment, computers used by patients or caregivers can be dummy terminals as long as the digital treatment program is being executed on a different computer or server.

This digital treatment program and method can have advantages over in-person therapy sessions. The use of an online system systemizes or standardizes many practices that can be skipped or shortchanged in an in-person setting. For example, the method described above is rigorous about administering pre-and post treatment assessments of the patient's condition and progress. This allows rigour about only allowing patients who are in the most appropriate mindset to most benefit from the treatment to access the treatment. It is also rigorous about allowing the patient control over when to voluntarily pause treatment to access grounding tools, and then return to the treatment. The patient will persevere with the treatment, improving adherence rates compared to traditional talk therapies.

While this treatment protocol, methods, program, and apparatus have been discussed in the context of the treatment of trauma and PTSD, it is believed that the same protocol, methods, program and apparatus can also be used to treat other conditions, such as anxiety, depression or burnout, or vicarious trauma.

### Results

The methods described in this application have been tested in a research study in adults with PTSD. Preliminary results are found in Charts A, B, C, and D, and are currently being peer reviewed. In all cases, “Intervention” shows the assessment results for patients treated using the protocols, methods and programs disclosed herein, and “Control” are patients to whom only assessments were administered without the therapy protocol. There were 10 Control patients and 11 Intervention patients. Overall, participants who used the inventive process, and system (labelled Intervention in Charts A, B, C and D) showed significant improvements in their PTSD symptoms by approximately 59% (Chart A), distress levels by approximately 64% (Chart B), depression symptoms improved by approximately 41% (Chart C) and general anxiety by approximately 43% (Chart D). Significance is tested using Student's t-test. (The exact results may change if different statistical analyses are applied) These results are similar to results obtained through in-person therapy.

In Chart A, the intervention group showed an approximate 59% decrease in PTSD symptoms.

In Chart B, the intervention group showed an approximate 64% decrease in distress.

In Chart C, the intervention group showed an approximate 41% decrease in depression symptoms.

In Chart D, the intervention group showed an approximate 43% decrease in anxiety symptoms.

The foregoing description, for purposes of explanation, uses specific nomenclature to provide a thorough understanding of the invention. However, it will be apparent to one in the art that specific details are not required in order to practice the invention. Thus, the foregoing description of specific embodiments of the invention is presented for purposes of illustration and description. It is not intended to be exhaustive or to limit the invention to the precise methods, systems, or the like disclosed; many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed.

Accordingly, the scope of the invention shall be defined solely by the following claims and their equivalents.

What is claimed is:

1. A method for the administration of therapy to a patient supervised by a caregiver, comprising:
administering assessments to the patient to qualify them to undertake the treatment;
administering assessments to the patient to assess their pre-treatment condition;
taking a detailed history of traumatic events;
having the patient set up a grounding tools;
for a specific traumatic event:
administering a pre-treatment check of the patient's receptiveness to treatment, by administering a first assessment and a second assessment, and stopping treatment if a first predetermined threshold is not met on these assessments;
administering an eye movement treatment comprising a cursor moving left to right and back across a computer screen;
inquiring as to whether the patient has changed feelings from the treatment, and if the answer is yes administering a third assessment, and if the third assessment meets a second predetermined threshold, notifying the caregiver;
where at all times after the step of setting up grounding tools, the patient has the ability to exit the treatment method and access grounding tools, and optionally can return to the treatment method after accessing the grounding tools.

2. The method of claim 2, further comprising:
for the specific traumatic event:
administering an exposure therapy pre-treatment check of the patient's receptiveness to treatment, by administering a fourth assessment and a fifth assessment, and stopping treatment if a third predetermined threshold is not met on these assessments;
administering an exposure therapy treatment comprising the patient recording their thoughts regarding the traumatic event;
inquiring as to whether the patient has changed feelings from the exposure therapy treatment, and if the answer is yes administering a sixth assessment, and if the sixth assessment meets a fourth predetermined threshold, notifying the caregiver;
where during the steps of administering an exposure treatment, and inquiring as to whether the patient has changed feelings from the exposure therapy treatment, the patient has the ability to exit the treatment method and access grounding tools, and optionally can return to the treatment method after accessing the grounding tools.

3. The method of claim 1, further comprising repeating the steps of administering a pre-treatment check; administering an eye movement treatment, and inquiring as to whether the patient has changed feelings from the treatment, six to eight times.

4. The method of claim 2, further comprising: repeating the steps of administering a pre-treatment check; administering an eye movement treatment, and inquiring as to whether the patient has changed feelings from the treatment, six to eight times; and
repeating the steps of administering an exposure therapy pre-treatment check;
administering an exposure treatment, and inquiring as to whether the patient has changed feelings from the exposure therapy treatment, three to five times.

5. The method of claim 4, where the second, third, fifth and sixth assessments are SUDS assessments.

6. The method of claim 5, where the first and fourth assessments are PCL assessments.

7. The method of claim 4, where the patient and caregiver are remote.

8. A system for the administration of therapy to a patient supervised by a caregiver, comprising:
a first computing device, the first computing device being configured to:
administer assessments to the patient to qualify them to undertake the treatment;
administer assessments to the patient to assess their pre-treatment condition;
take a detailed history of traumatic events;
have the patient set up a grounding tools;
for a specific traumatic event:
administer a pre-treatment check of the patient's receptiveness to treatment, by administering a first assessment and a second assessment, and stopping treatment if a first predetermined threshold is not met on these assessments;
administer eye movement treatment comprising a cursor moving left to right and back across a computer screen;
inquire as to whether the patient has changed feelings from the treatment, and if the answer is yes administering a third assessment, and if the third assessment meets a second predetermined threshold, notifying the caregiver;
where at all times after the step of setting up grounding tools, the patient has the ability to exit the treatment method and access grounding tools, and optionally can return to the treatment method after accessing the grounding tools.

9. The system of claim 8, where the first computing device is further configured to:
for the specific traumatic event:
administer an exposure therapy pre-treatment check of the patient's receptiveness to treatment, by administering a fourth assessment and a fifth assessment, and stopping treatment if a third predetermined threshold is not met on these assessments;
administer an exposure therapy treatment comprising the patient recording their thoughts regarding the traumatic event;
inquire as to whether the patient has changed feelings from the exposure therapy treatment, and if the answer is yes administering a sixth assessment, and if the sixth assessment meets a fourth predetermined threshold, notifying the caregiver;
where during the steps of administering an exposure treatment, and inquiring as to whether the patient has changed feelings from the exposure therapy treatment, the patient has the ability to exit the treatment method and access grounding tools, and optionally can return to the treatment method after accessing the grounding tools.

10. The system of claim 8, further comprising the first computing device being configured to: repeat the steps of administering a pre-treatment check; administer an eye movement treatment, and inquire as to whether the patient has changed feelings from the treatment, six to eight times.

11. The system of claim 9, further comprising the first computing device being configured to: repeat the steps of administering a pre-treatment check; administer an eye movement treatment, and inquire as to whether the patient has changed feelings from the treatment, six to eight times;
and
repeat the steps of administering an exposure therapy pre-treatment check;
administering an exposure treatment, and inquiring as to whether the patient has changed feelings from the exposure therapy treatment, three to five times.

12. The system of claim 11, where the second, third, fifth and sixth assessments are SUDS assessments.

13. The system of claim 12, where the first and fourth assessments are PCL assessments.

14. The system of claim 11, where the first computing device is configured by the first computing device executing a program, and further comprising a second computing device, and the patient accessing the program being executed on the first computing device by communication between the second computing device and the first computing device.

15. The system of claim 14, where the first computing device is configured by the first computing device executing a program, and further comprising a third computing device, and the caregiver accessing the program being executed on the first computing device by communication between the third computing device and the first computing device.
